(19) United States
(12) Patent Application Publication
(10) Pub. No.: US 2008/0167638 A1
(43) Pub. Date: Jul. 10, 2008

(54) Method and Apparatus for Delivering Pharmaceuticals Based on Real-Time Monitoring of Tissue States

(75) Inventors: Guillermo A. Cecchi, New York, NY (US); James R. Kozloski, New Fairfield, CT (US); Ravishankar Rao, Elmsford, NY (US)

Correspondence Address: CANTOR COLBURN LLP-IBM YORKTOWN, 20 Church Street, 22nd Floor, Hartford, CT, 06103, US

(73) Assignee: INTERNATIONAL BUSINESS MACHINES CORPORATION, Armonk, NY (US)

(21) Appl. No.: 11/619,688

(22) Filed: Jan. 4, 2007

(51) Int. Cl. A61M 5/142; A61M 31/00

(52) U.S. Cl. 604/503; 604/151

(57) ABSTRACT

A method and apparatus for delivering a drug to a bodily tissue, measuring the interaction between the drug and a specific tissue in real-time, and adjusting the rate of drug delivery in real-time in order to achieve a desired state of drug-tissue interaction are disclosed herein.

### TRADEMARKS

IBM® is a registered trademark of International Business Machines Corporation, Armonk, N.Y., U.S.A. Other names used herein may be registered trademarks, trademarks or product names of International Business Machines Corporation or other companies.

### BACKGROUND OF THE INVENTION

1. Field of the Invention

This invention relates to an apparatus, system, and method for delivering pharmaceuticals based on real-time monitoring of a bodily tissue, and particularly to an apparatus, system, and method for delivering a drug to a bodily tissue, measuring the interaction between the drug and the bodily tissue in real-time, and adjusting the rate of drug delivery in real-time in order to achieve a desired state of drug-tissue interaction.

2. Description of Background

The problem of delivering drugs to specific bodily tissues and achieving specific tissue-drug interactions is closely related to the problem of adjusting the dose of a prescribed medication to treat illness or enhance bodily function. Often the prescribed dose is intended to ensure the pharmaceutical achieves some desired concentration within the tissue of interest during some prescribed time period. In the case of PET imaging of the brain, a prescribed steady state concentration in some reference tissue must be achieved before further measurements or investigations can be made. The time and concentration level at which this steady state is eventually achieved is a function of the infusion rate of the drug. This infusion rate must therefore be calculated in advance of PET scanning by drawing multiple blood samples during a preliminary exposure to the pharmaceutical. This poses a problem for human therapies and measurements in that it can involve multiple infusions of a biologically active pharmaceutical during a preliminary “trial-and-error” phase. While drawing blood samples and performing said measurements cannot be easily expedited, the time to measure concentrations of the drug in the tissue of interest or some reference tissue using medical imaging technology such as PET is limited primarily by the rate of computation applied to the raw data once it is collected.

Closed-loop systems for pharmaceutical delivery have been widely used in other contexts. One example is the insulin pump. In this system, a continual monitoring of blood glucose levels controls delivery of insulin to the body in order to maintain it within a healthy range. These systems do not make use of high-performance computing because the analysis of the measured data does not require it in order to provide a real-time feedback signal.

The invention disclosed herein monitors drug-tissue interaction by electrical or chemical monitoring devices, and delivers pharmaceuticals to the body in a closed-loop in order to achieve a particular tissue state and therapeutic result. Real-time monitoring of tissue states and feedback to the drug delivery system are achieved by high-performance computing.

### SUMMARY OF THE INVENTION

The shortcomings of the prior art are overcome and additional advantages are provided through the provision of a method and apparatus for delivering a drug to a bodily tissue based on real-time monitoring of the state of the bodily tissue.

The method comprises delivering a drug to a patient; measuring an interaction between the drug and a bodily tissue in real-time; and adjusting a rate of drug delivery to the patient in real-time based on the measurement.

The apparatus comprises a delivery mechanism for delivering a drug to a patient; a measuring device for measuring a state of a bodily tissue; and an information handling device for processing the measurement and computing an amount of drug to be delivered given a current tissue state measurement and a desired tissue state.

System and computer program products corresponding to the above-summarized method are also described and claimed herein.

Additional features and advantages are realized through the techniques of the present invention. Other embodiments and aspects of the invention are described in detail herein and are considered a part of the claimed invention. For a better understanding of the invention with advantages and features, refer to the description and to the drawings.

### TECHNICAL EFFECTS

As a result of the summarized invention, technically we have achieved a solution which monitors tissue state in real-time by electrical or chemical monitoring devices, and delivers pharmaceuticals to the body in a closed-loop in order to achieve a particular tissue state and therapeutic result using high-performance computing.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 illustrates one example of an apparatus that implements the invention, comprising a measurement device, a real-time data processor, a control system, and a drug infusion device.

The detailed description explains the preferred embodiments of the invention, together with advantages and features, by way of example with reference to the drawings.

### DETAILED DESCRIPTION OF THE INVENTION

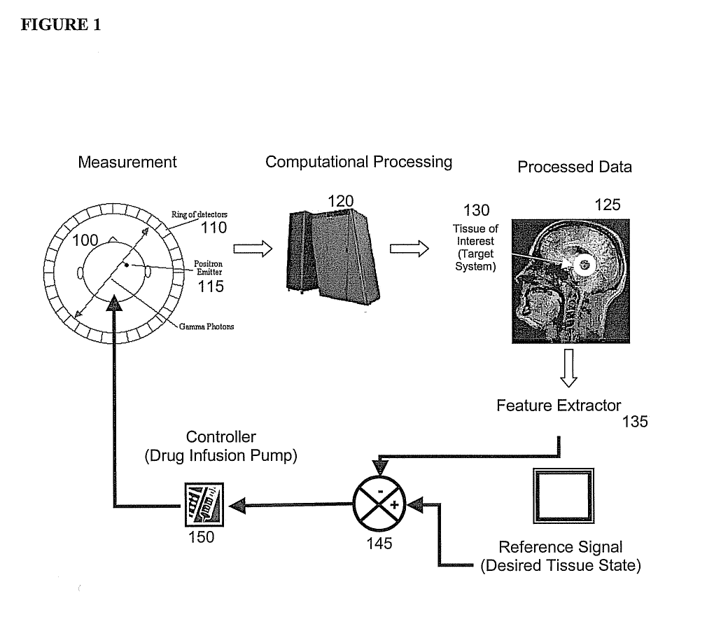

Turning now to the drawings in greater detail, it will be seen that in FIG. 1 there is an example of an apparatus that implements the method. The embodiment in FIG. 1 comprises a measuring device for the state of a brain tissue, an information handling device for computing the best drug delivery parameters given the current and desired brain tissue states, and a delivery mechanism for a drug. Based on analysis of individual and population data, an optimal trajectory to the desired brain tissue state given the current recording is computed in the space of pharmaceutical delivery. The drug delivery mechanism then delivers the therapy accordingly.

The drug delivery mechanism can be, for example, a drug pump with access to the blood stream.

The measuring device may be an electrical monitor or a chemical monitor. The electrical monitor can monitor brain activity via external contacts to the scalp, subcutaneous contacts, or via implants. The chemical monitor can be, for example an implanted biosensor or a positron emission tomography (PET) scanner. PET has been used successfully as a chemical monitor. For example, PET was used to compare binding of a dopamine-2 receptor tracer to the caudate nucleus following amphetamine challenge in healthy subjects and in untreated subjects with schizophrenia (A Abi-Dargham, et al., “Increased Striatal Dopamine Transmission in Schizophrenia: Confirmation in a Second Cohort” Am J Psychiatry. (1998) 155(6):761-7).

The method relies upon a measurement taken from a patient (100), for example, positron emission from a radioactively labeled drug compound (115) using a measurement device, such as a ring of position emission detectors (110). Measurements of this sort rely upon extensive computational processing (120) in order to create a usable set of data, for example an image-based reconstruction of the measurements (125), including specific local activity levels within tissues of interest (130). The current method relies upon high-performance computing in order to minimize the time it takes to process measurements into a usable form for the purpose of controlling the drug delivery system. Computer simulations indicate the method is workable under conditions of a clinical setting.

The drug delivery system extracts features from the specific brain tissue measurements (135) and compares them to some pre-determined reference signal (140) in order to compute a control signal (145). The control signal may be computed using a standard algorithm, such as Proportional, Integral, Differential or some combination thereof, such as PID (Proportional-Integral-Differential). These methods are described in Feedback Control of Computing Systems by Joseph L. Hellerstein, John Wiley & Sons Inc, 2004, p. 320. The reference signal reflects the desired state of the tissue of interest, for instance a steady-state drug concentration in this tissue intended to create some therapeutic outcome. A physician may, for example, select the tissue of interest, and prescribe that the drug achieve some desired level of tissue interaction in this region as estimated from the PET image data using any data analysis techniques that may be appropriate, for example regression methods or artificial neural networks. The analysis is intended to establish a mapping from the PET image to some measure of the interaction in the tissue of interest. The analysis may further project how this measure will change; for example, it may predict when and at what level a measure will achieve a steady state in the tissue of interest. The predictive model used in the latter analysis can be based on recent measures from the tissue, or on historical measurements from a population of patients and brain areas under similar conditions. It is the system's responsibility then to control drug infusion (150) into the patient (100) in order to bring about the physician's prescription (for example, a particular steady state value). The current method relies upon high-performance computing to achieve rapid measurement, analysis, feature extraction, and computation of the control signal controlling the drug delivery system.

The system is therefore a closed-loop control system operating in real-time in order to affect an outcome in a dynamical system using a drug delivery system.

Any standard control algorithm, such as a Proportional-Integral-Differential (PID) control algorithm, is applicable to an implementation of the method. The choice of control algorithm should be based on the control objective. The control objective can be, for example, a specific steady state value, and/or minimal settling time, and/or avoidance of overshoot, but is not limited thereto. The method described is not specific to any one control objective or control algorithm, but assumes an appropriate algorithm will be chosen depending on the objective.

The advantages of the invention disclosed herein are that it allows direct monitoring of binding, steady state, and dynamical interactions of the infused drug with specific bodily tissues, for example specific brain tissues; that it allows measurements to be made in real-time, thus permitting control systems to adjust the rate of drug-delivery on this time scale and thus exercise greater control over the drug-tissue interaction; and its accuracy resulting from directly quantifying drug-tissue interactions.

The capabilities of the present invention can be implemented in software, firmware, hardware or some combination thereof.

As one example, one or more aspects of the present invention can be included in an article of manufacture (e.g., one or more computer program products) having, for instance, computer usable media. The media has embodied therein, for instance, computer readable program code means for providing and facilitating the capabilities of the present invention. The article of manufacture can be included as a part of a computer system or sold separately.

Additionally, at least one program storage device readable by a machine, tangibly embodying at least one program of instructions executable by the machine to perform the capabilities of the present invention can be provided.

There may be many variations to the steps (or operations) described herein without departing from the spirit of the invention. For instance, the steps may be performed in a differing order, or steps may be added, deleted or modified. All of these variations are considered a part of the claimed invention.

While the preferred embodiment to the invention has been described, it will be understood that those skilled in the art, both now and in the future, may make various improvements and enhancements which fall within the scope of the claims which follow. These claims should be construed to maintain the proper protection for the invention first described.

What is claimed is:

1. A method for delivering a drug to a bodily tissue, comprising
delivering a drug to a patient;
measuring an interaction between the drug and a bodily tissue in real-time; and
adjusting a rate of drug delivery to the patient in real-time based on the measurement.

2. The method of claim 1 wherein measuring the interaction comprises estimating the steady state concentration of the drug in the bodily tissue at a given delivery rate.

3. The method of claim 2 wherein measuring the interaction comprises in vivo imaging of the tissue by positron emission tomography.

4. The method of claim 1, wherein adjusting the rate comprises:
extracting a feature from the measured interaction;
comparing the feature to a reference state; and
computing a control signal based on the comparison to adjust the rate of drug delivery.

5. The method of claim 4, wherein analyzing the measured interaction, extracting the feature, comparing the feature to the reference state, and computing the control signal are performed by high-performance computing.

6. The method of claim 5, wherein the control signal is computed using a proportional-integral-differential control algorithm.

7. An apparatus for delivering a drug to a bodily tissue, comprising:
a delivery mechanism for a drug;
a measuring device for measuring a state of a bodily tissue; and
an information handling device for computing an amount of drug to be delivered given a current tissue state measurement and a desired tissue state.

8. The apparatus of claim 7, wherein the measuring device is an electrical or a chemical monitoring device.

9. The apparatus of claim 7, wherein the delivery mechanism is a drug pump with access to a patient's blood stream.

10. The apparatus of claim 7, wherein the information handling device is a high-performance computing device.

11. The apparatus of claim 7, wherein the information handling device uses a proportional-integral-differential control algorithm to compute the amount of drug delivered.
